(19) United States
(12) Patent Application Publication
(10) Pub. No.: US 2002/0125164 A1
(43) Pub. Date: Sep. 12, 2002

(54) Personal medical dispensing card

(76) Inventor: Oscar M. Bassinson, Tarzana, CA (US)

Correspondence Address: REINHART BOERNER VAN DEUREN S.C., ATTN: LINDA GABRIEL, DOCKET COORDINATOR, 1000 NORTH WATER STREET, SUITE 2100, MILWAUKEE, WI, 53202, US

(21) Appl. No.: 10/027,565

(22) Filed: Dec. 21, 2001

Related U.S. Application Data
(60) Provisional application No. 60/259,275, filed on Dec. 21, 2000.

(51) Int. Cl. A61L 15/00

(52) U.S. Cl. 206/438

(57) ABSTRACT

A personal medication dispensing card capable of being retained by an individual much in the same manner as a conventional credit card. The personal medication dispensing card is provided with a shallow recess in a surface thereof for containing a powdered or granulized form of the medication or other substance to be dispensed in that recess. A removable cover sheet is disposed over the recess and holds the contents in place. The medication or other substance can also be located on a surface of the card and covered by a removable cover sheet. When a user desires to consume that medication, the user merely removes the removable cover sheet and typically licks the medication contained in the recess.

### CROSS REFERENCE TO RELATED APPLICATION

This application claims priority of provisional application No. 60/259,275, filed on Dec. 21, 2000.

### BACKGROUND OF THE INVENTION

The invention relates to medication packages, and more particularly, to a personal medication dispensing device of a size that is similar to that of a credit card or the like, which can be carried in a wallet of an individual or a purse of an individual.

There are numerous instances in which individuals are periodically required to take medication on a regular basis. As a simple example, diabetic persons are required to take certain medications periodically in order to keep blood sugar levels under control. In other cases, individuals such as those with coronary problems, are required to constantly carry medication such as nitroglycerin in the event of a type of coronary interruption.

One frequently used technique for carrying medication is in the form of so-called medicine boxes or tablet boxes normally of relatively small shape and size which can be carried in a pocket or a purse. These boxes are usually formed of a transparent plastic material so that one may observe the contents.

Another frequently used technique for carrying personal medication is that of the use of so-called plastic baggy in which an individual places a desired quantity of medication in that plastic bag and seals same. Both techniques are somewhat effective when the individual remembers to insert medication either within the pill box or the bag or the like and carry same on his or her person. However, frequently the individual will forget this step. In some cases, forgetting the medication can give rise to a life threatening situation and, therefore, becomes a very serious problem. It would be desirable to provide a means for insuring that an individual always has medication available.

In this present society, credit cards and personal identification cards have become so common place that almost every individual carries some type of card-like device, usually plastic or paperboard cards having a size roughly of about 3 and ⅜ inches by 2 and ⅛ inches. Credit card-sized medication packages have been proposed by the prior art. One such medication package, is disclosed in U.S. Pat. No. 4,889,236 which was issued to Ralph Bartell, et al. This package is a rigid, blister-pack medication dispensing card designed to contain medication which must be taken on a calendar day schedule. The card has a plurality of chambers for containing pills or tablets. Although this package has peripheral dimensions that are similar to those of a credit card, the package must be about five to ten times thicker than a credit card to accommodate the tablets or pills to be dispensed.

Another credit card-sized medication package is disclosed in U.S. Pat. No. 3,958,690 which was issued to Robert W. Gee, Sr. This package includes a foldable card, slidably received within an envelope and a frame folded within a card, defining a space for containing a medication, such as a conventional packet of sugar for use by a diabetic. The open frame construction of this package renders the package suitable only for prepackaged medications, such as sugar.

### SUMMARY OF THE INVENTION

It is, therefore, one of the primary objects of the present invention to provide a personal card-like device having means for containing a medication capable of being dispensed.

It is another object of the present invention to provide a personal card-like device of the type stated in which the medication can be licked from a recess in this card-like device or from a surface on the card-like device.

Another object of the present invention to provide a method of dispensing a medication.

With the above and other objects in view, my invention resides in the novel features or form, construction, arrangement and combination of parts and components presently described and pointed out in the claims.

The present invention provides a medication dispensing card adapted for carrying a medication. The medication dispensing card includes a card-like base formed of a relatively rigid material, a surface on the base containing a medication thereon. The medication is capable of being soluably removable by a user upon contact with a portion of the user's mouth. The medication dispensing card further includes a removable cover disposed over the medication, the cover being removable to enable a user to engage the medication. Medication is in powder form and includes a binding agent.

Further in accordance with the invention, there is provided a personal medication dispensing card having a size and shape similar to those of a credit card and adapted for carrying a personal medication with an individual. The personal medication dispensing card includes a card-like base having a size and shape similar to a credit card and being formed of a relatively rigid material. The base has a surface with at least one shallow recess therein and a medication is disposed in the recess on the base. The personal medication dispensing card further includes a removable cover disposed on the base overlying the recess to enclose the medication on the base. The cover is at least partially removable by a user to provide access to the medication in the recess.

In one embodiment, the medication is in powder form and includes a binding agent. In another embodiment, the medication is in powder form and includes a gelling substance for retaining the medication on the surface.

In accordance with a further aspect of the invention, there is provided a method of delivering a medication, the method comprising the steps of packaging the medication in a medication dispensing card including applying the medication to a surface of a base of the card; and applying a removable cover to the base to enclose the medication on the base; and dispensing the medication including at least partially removing the cover from the base to expose the medication to a user; and soluably removing the medication from the surface.

Thus, the present invention therefore provides a card-like device which is provided with a recess capable of holding the medication therein. In order to preclude the medication from leaking out of the recess, even when covered by a removable cover sheet, the medication is introduced into a granular form which is retentively held within the recess by a gelling agent or like member. In an alternative embodiment, the medication can be placed on a surface of the card, without a recess, particularly if a binding agent is used in particulate medication. Thus, when an individual has a need to consume that medication, the individual can typically lick the surface of the medication. The moisture from that individual's mouth will thereupon dissolve the medication and allow the individual to consume same.

Any form of removable cover may be provided for purposes of covering the medication. In one embodiment, the cover comprises a thin, pliable plastic film.

The personal medication dispensing card according to the invention is a convenient way for a consumer to carry medication in a wallet or purse for a determined length of time. Most individuals carry a wallet or purse much of the time so that the medication carried therein will be available to the consumer whenever it is needed, especially in an emergency situation. However, use of the personal medication dispensing card for delivering medication is not limited to emergency situations and can provide a convenient vehicle for dispensing a dose of medication on a daily or other periodic basis, and particularly in situations in which a consumer would prefer to lick the medication off a card-like device rather than swallow a pill or tablet.

With reference to the foregoing, the present invention can also include a method of using a card surface to deliver a medication. Such a method includes (1) providing a card surface having applied thereto a medication, and (2) contacting the card surface to remove the medication therefrom. While the medication can be frictionally removed from the card, preferred contact soluably removes the medication, such removal as can be achieved either by oral contact therewith or by contact with a suitable solvent. Other aspects relating to this inventive method are as described more fully herein. In particular, reference is made to various card articles illustrated in the figures and the description regarding medication applied thereto.

Accordingly, the present invention further includes a method for achieving a therapeutic effect in a subject. Such a method includes administering an effective amount of a medication including, but not limited to aspirin, vitamin K, nitroglycerine, an antihistamine, insulin, sugar, a snake bite antidote, epinephrine, heparin, demetrex, zofran, dramamine, scopolamine, a nicotine replacement medication, pepcid, hydergine, compazine and combinations of said medications. Administration is achieved by removing from a card surface such a medication applied thereto. In preferred embodiments, such a medication is applied as described more fully above, or as would otherwise be understood by those skilled in the art made aware of this invention Such skill could also readily optimize effective amounts, dosages and/or administration regimens for each such medication. As is well known, the specific dosage for a given subject under specific conditions and for a specific medical condition will routinely vary, but determination of an optimum amount can be readily accomplished by routine procedures, without undue experimentation.

Various preferred embodiments of such a method can, optionally, include use of a pharmaceutically-acceptable binder composition in conjunction with such a medication. Such binders and formulations thereof are, as would be well known to those skilled in the art, dependent upon a particular medication, particle size and—in the context of this invention—the card/article surface and/or configuration employed for administration. Nonetheless, such methods and formulations of this invention contemplate binders including, but not limited to, biodegradable polymers, polymers derived from acrylic acid and substitute derivatives thereof, cyclodextrins, lipids/lipoproteins and the like. For purposes of illustration and without limitation thereto, preferred binder compositions include locust green gum, xanthan gum, propylene glycol, methylcellulose, carboxymethylcellulose and eurothane. The latter composition is especially useful for applications of the present invention in that it provides an adhesive quality to the medication, soluably removed upon contact with moisture, saliva or another suitable solvent.

As illustrated by the foregoing, the present invention can also include a therapeutic formulation including a medication such as, but not limited to, any of those described elsewhere herein, such medication as applied to a card surface and carried therewith. In preferred embodiments, such a medication further includes a binder of the sort described above. However, in various other embodiments, the medication can be applied to such an article, as described above, for subsequent administration. Accordingly, with respect to the foregoing discussion concerning therapeutically effective amounts or concentrations, the present invention can also include a dosage administration article, the medication applied thereto in an amount sufficient to provide a therapeutic dose thereof by removal from the article.

This invention has many other advantages and has other purposes which may be made more clearly apparent from a consideration of the forms in which it may be embodied. These forms are shown in the drawings forming a part of and accompanying the present specification. They will now be described in detail for purposes of illustrating the general principles of the invention. However, it is to be understood that the following detailed description and the accompanying drawings are not to be taken in a limiting sense.

### BRIEF DESCRIPTION OF THE DRAWINGS

The features of the present invention which are believed to be novel are set forth with particularity in the appended claims. The invention, together with the further objects and advantages thereof, may best be understood by reference to the following description taken in conjunction with the accompanying drawings, wherein like reference numerals identify like elements, and wherein:

FIG. 1 is a perspective view of a personal medication dispensing card in accordance with one embodiment of the present invention;

FIG. 2 is an exploded perspective view of the personal medication dispensing card of FIG. 1, with the medication partially removed to show a portion of a medication containing recess of the personal medication dispensing card;

FIG. 3 is a perspective view of a personal medication dispensing card in accordance with a further embodiment of the invention, and which includes a plurality of medication containing recesses;

FIG. 4 is a perspective view of a personal medication dispensing card in accordance with another embodiment of the invention, and which includes a removable cover strip disposed over a medication containing recess thereof;

FIG. 5 is a perspective view of the personal medication dispensing card of FIG. 4, with the cover strip shown partially removed;

FIG. 6 is an enlarged, vertical section view taken along the line 6-6 of FIG. 5;

FIG. 7 is a perspective view of a personal medication dispensing card similar to that of FIG. 4 wherein the medication containing recess is generally oval in shape;

FIG. 8 is a perspective view of a personal medication dispensing card in accordance with another embodiment of the invention wherein a medication is disposed on the surface of the card; and

FIG. 9 is an enlarged, vertical section view taken along the line 9-9 of FIG. 8.

### DETAILED DESCRIPTION OF A PREFERRED EMBODIMENT

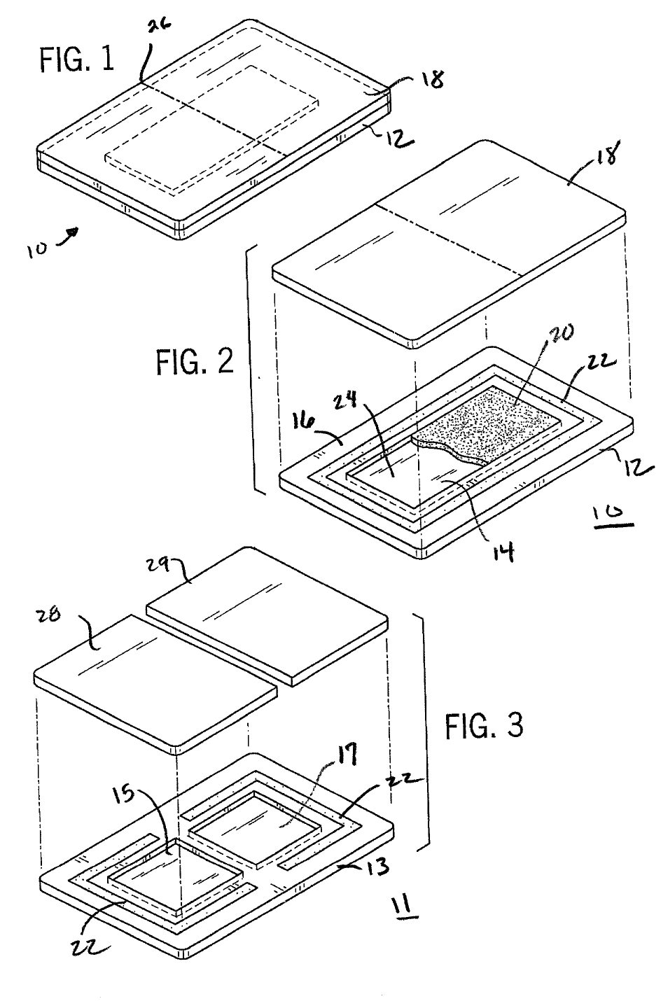

Referring FIGS. 1 and 2 of the drawings, there is shown a personal medication dispensing card 10 in accordance with one embodiment of the invention. The personal medication dispensing card 10 is similar in size and shape to a conventional credit card and includes a base 12 having a shallow recess 14 in the upper surface 16 thereof. The personal medication dispensing card 10 also includes a removable top cover plate 18 which covers medication 20 contained in the recess 14.

The term “medication” is used in a broad sense to essentially incorporate any type of consumable item, for example, vitamin tablets, homeopathic remedies and the like. The medication can include, but is not limited to aspirin, vitamin K, nitroglycerine, an antihistamine, insulin, sugar, a snake bite antidote, epinephrine, heparin, demetrex (generic: sumatriptan), zofran (generic: ondansetron), dramamine, scopolamine, a nicotine replacement medication, pepcid (generic: proclorperazine), hydergine, compazine (generic: sumatriptan) and combinations of said medications. Thus, the term “medication” is not specifically limited to either an over the counter specific medicine or a prescribed medicine, but rather used in this broad sense. Moreover, although the personal medication dispensing card provided by the invention is described with reference to an application for dispensing medication to humans, the invention can find application in emergency, daily or periodic veterinary uses for animals that obviously can lick a medication rather than swallow a pill. In the case of veterinary uses, the size of the card can be increased, and different shapes can be used, if necessary.

The base 12 is of a plastic material, such as polyvinyl chloride. In one embodiment, the base 12 is about 3 and ⅜inches in length by 2 and ⅛inches width and about 0.040 inches to 0.060 inches thick. The shallow recess 14 covers only a portion of the upper surface 16 of the base 12 and is about 2 and ⅞ inches in length by 1 and ⅝ inches in width and about 0.030 inches deep.

In one preferred embodiment, the medication 20 is deposited in a particulate or powder state in the recess 14 and maintained in the recess 14 by the top cover plate 18. Typically, the medication substantially fills the recess and is flush with the upper surface 16 of the base 12. The medication can be dispensed by removing the top cover plate 18, for example, and pouring the medication into one hand. In another embodiment, the medication, in a particulate or powder state, is mixed with a binder forming a slurry. Methods and formulations of this invention contemplate binders including, but not limited to, biodegradable polymers, polymers derived from acrylic acid and substitute derivatives thereof, cyclodextrins, lipids/lipoproteins and the like. For purposes of illustration and without limitation thereto, preferred binder compositions include locust green gum, xanthan gum, propylene glycol, methyl cellulose, carboxymethyl cellulose and eurothane. The latter composition is especially useful for applications of the present invention in that it provides an adhesive quality to the medication, soluably removed upon contact with moisture, saliva or another suitable solvent. The medication is applied to the surface 24 of the recess 14 in liquid form and then dried on the surface of the recess. This allows the medication to be distributed over substantially the entire surface 24 of the recess.

Alternatively, the medication can be prepared with a gelling agent to form a paste-like substance to facilitate application of the medication to the surface 24. Gelling substances suitable for this application include carboxymethyl cellulose, methyl cellulose, eurothane, locust green gum, propylene glycol, and Xanthan gum. The gelled medication can be dried or in a tacky state. In these embodiments, the medication can be soluably removed from the card by removing the top cover plate 18 and introducing a solvent, such as water, saliva, etc. to dissolve the medication into a solution, facilitating removal of the medication from the card. For example, the card can be placed in a vessel containing water or some other solvent, of the medication can be licked out of the recess by the individual for whom the medication is intended or the vessel with water and the card can be swallowed.

The top cover plate 18 also can be formed of a relatively thin, plastic or paperboard material. The top cover plate 18 can be clear, translucent or opaque. In one embodiment, the top cover plate 18 is about 0.020 inches to 0.030 inches thick and has peripheral dimensions substantially the same as those for the base 12. The top cover plate 18 can be secured to the base 12 by any suitable means including a permanent or a releasable adhesive 22, and the like, or some type of mechanical fasteners. The top cover plate 18 lies substantially flat on the upper surface 16 of the base. When a permanent adhesive is used, the top cover plate 18 can include a score line 26, allowing the medication to be accessed by breaking the top cover plate by flexing the card. When a releasable adhesive is used, a user can merely remove the top cover plate 18 by inserting a finger tip, or finger nail, at one end, for example, and prying the top cover plate 18 off of the base to obtain access to the medication 20 contained in the recess 14, allowing the medication to be poured into one hand, when the medication is in particulate form, or to be soluably removed when the medication is dried on or otherwise applied to the surface of the recess. The poured particulate can also be poured into water for drinking. Preferably, the adhesive is disposed on the base 12. However, the adhesive can be disposed on the inner surface of the top cover plate 18.

Although the base 12 of the personal medication dispensing card 10 illustrated in FIG. 1 includes a single recess 14, it is apparent that the base can include two or more recesses, each having a medication applied to the surface of the recess. For example, with reference to FIG. 3, there is shown a personal medication dispensing card 11 which is similar to the personal medication dispensing card 10 illustrated in FIG. 1, but which includes two recesses 15 and 17 in the base 13. The medications contained in the recesses 15 and 17 can be the same medication or different medications. In such embodiment, two individually removable top cover members 28 and 29 are provided to allow for access to a single medication containing compartment at a time. The top cover members 28 and 29 are maintained on the base by adhesive 22 which can extend around three sides of the recesses as illustrated, or extend around all four sides of each recess in the manner of top cover plate 18 (FIG. 1)

Referring to FIGS. 4-6, in accordance with a further embodiment of the invention, a personal medication dispensing card 30 includes a base 12, the dimensions of which can be the same as those of personal medication dispensing card 10 shown in FIGS. 1-2, and including one or more recesses, such as recess 14 for containing a medication 20. The medication can be applied to the surface of the recess 14 in liquid form and then dried on the surface of the recess, in the manner described above with reference to FIGS. 1 and 2. Alternatively, the medication can be prepared with a gelling agent, forming a paste-like substance that can be spread onto the surface of the recess, preferably substantially filling the recess 14 so that the upper surface of the medication 20 is substantially flush with the upper surface of the base 12.

In this embodiment, the personal medication dispensing card 30 includes a removable cover member 32 in the form of a pliable, plastic tear-away strip. The cover member 32 can be clear, translucent or opaque. In one embodiment, the cover strip 32 is a thin film of plastic approximately 0.005 inches thick. The cover strip 32 is held in place by an adhesive 34 and can be adhesively sealed around the periphery of the recess 14 as shown in FIG. 4. The adhesive preferably is disposed on the base 12, but the adhesive can be disposed on the inner surface of the cover strip 32. Although for purposes of illustration the outer periphery of the cover strip 32 is shown raised above the upper surface of the base 12 by the adhesive 34, the cover strip 32, including its outer edge portions, lies substantially flat on the upper surface 16 of the base 12. In the embodiment illustrated in FIGS. 4-6, the adhesive 34 is disposed on the base 12, adjacent to the recess. This arrangement is preferred when the medication is disposed in the recess 14 in powder form, without a binder or gelling agent. A plurality of tear-away strips can be provided, one for each recess, when the base includes a plurality of recesses, enabling access to the recesses individually.

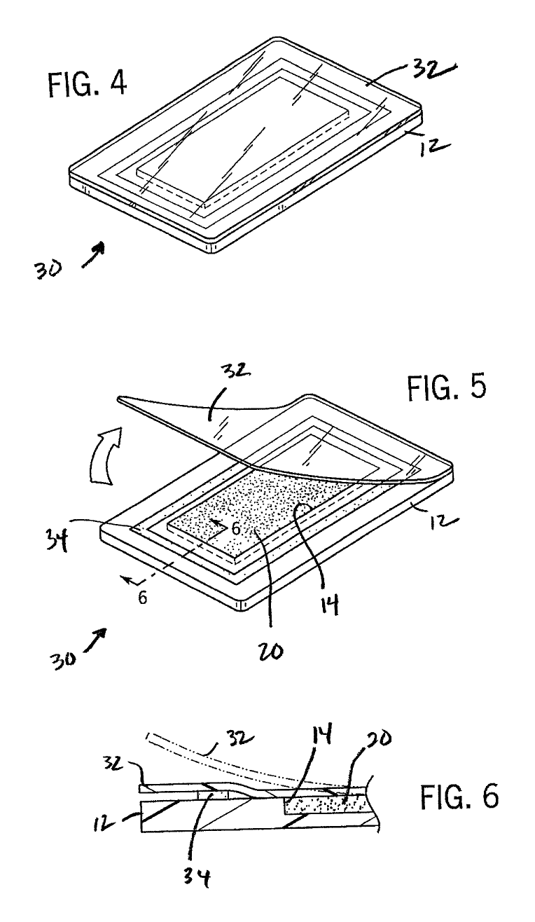

Referring to FIG. 5, a portion of the cover strip 32 has been pulled up from the surface 16 of the base 12, exposing the medication 20 for access by the user. The cover strip 32 can be peeled back to an almost completely removed position, allowing a user to literally engage his or her tongue with the surface of the medication 20 in the recess 14. In this case, the user is able to literally lick the medication out of the recess 14 of the base, soluably removing the medication from the recess.

Although the recess 14 is generally rectangular in shape, it is apparent that the shape of the recess can be any geometrical shape. For example, FIG. 7 illustrates a personal medication dispensing card 36 in which the recess 37 in the base 38 is generally oval in shape.

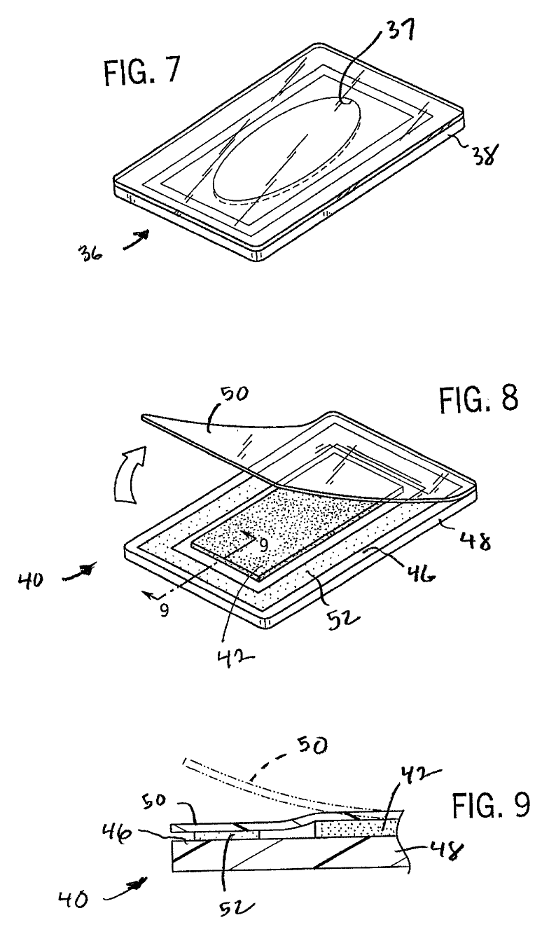

Referring to FIGS. 8 and 9, in accordance with another embodiment of a personal medication dispensing card 40 provided by the invention, the medication 42 is disposed on a planar upper surface 46 of the base 48. The medication can be applied to the surface 46 of the base 48 in liquid form and then dried on the surface 46 of the base or the medication can be prepared with a gelling substance and applied to the surface 46. However, the base 48 can contain a recess in the manner of personal medication dispensing card 30 (FIG. 5), with the medication applied to the surface of the recess. The base 48 can have the same peripheral dimensions as base 12 in FIG. 1, for example. The medication 42 is covered by a cover strip 50 which can be a thin film of a pliable plastic material which is approximately 0.005 inches thick. The cover strip 50 can be clear, translucent or opaque. The cover strip 50 is sealed by releasable adhesive 52 along the peripheral edge of the base 48. The adhesive preferably is disposed on the base 48, but can be disposed on the inner surface of the cover strip 50. The cover strip 50 can be peeled back to an almost completely removed position as shown in FIG. 8, allowing a user to literally engage his or her tongue with the surface of the medication 42 on the surface 46. In this case, the user is thereupon able to literally lick the medication off of the surface of the base, or out of the recess when the base includes a recess.

It should be understood in connection with the present invention that it is not always necessary to maintain the medication in a dry powder state and which is held together by a gum-like substance. Rather, the medication could actually exist in the form of a gelled state and would be conveniently gelled with a substance such as sodium carboxymethyl cellulose.

While preferred embodiments have been illustrated and described, it should be understood that changes and modifications can be made thereto without departing from the invention in its broadest aspects. Various features of the invention are defined in the following claims.

What is claimed is:

1. A medication dispensing card adapted for carrying a medication, said medication dispensing card comprising:
a card-like base formed of a relatively rigid material;
a surface on said base for containing a medication thereon and which is capable of being soluably removable by a user upon contact with a portion of the user's mouth; and
a removable cover disposed over said medication, said cover removable by a user to enable the user to engage the medication.

2. The medication dispensing card according to claim 1, wherein said medication is in powder form and includes a binding agent.

3. The medication dispensing card according to claim 1, wherein said medication is in powder form and includes a gelling substance for retaining said medication on said surface.

4. The medication dispensing card according to claim 1, wherein said base includes a shallow recess and said surface is located within said recess, said medication being applied to said surface.

5. A personal medication dispensing card having a size and shape similar to those of a credit card and adapted for carrying a personal medication with an individual, said personal medication dispensing card comprising:
a card-like base having a size and shape similar to a credit card and being formed of a relatively rigid material, said base having a surface;
a medication applied to said surface of said base; and
a removable cover disposed over said medication and removably secured to said base, said cover being removable by a user to enable the user to access said medication.

6. The personal medication dispensing card according to claim 5, wherein said medication is in powder form and includes a binding agent.

7. The personal medication dispensing card according to claim 5, wherein said medication is in powder form and includes a gelling substance for retaining said medication on said surface.

8. The personal medication dispensing card according to claim 5, wherein said base includes at least one shallow recess, said medication being disposed in said recess, covering substantially all of said surface.

9. The personal medication dispensing card according to claim 5, wherein said cover lies substantially flat on said surface of said base.

10. The personal medication dispensing card according to claim 5, wherein said cover is of a pliable material and i s disposed on said base in overlying relation with said recess, said cover being adapted to be peeled back from said recess to provide access to said medication.

11. The personal medication dispensing card according to claim 5, wherein said cover is adhesively sealed to said base about the periphery of said recess.

12. The personal medication dispensing card according to claim 5, wherein said cover is adhesively sealed to said base about the periphery of said base.

13. A personal medication dispensing card having a size and shape similar to those of a credit card and adapted for carrying a personal medication with an individual, said personal medication dispensing card comprising:
a card-like base having a size and shape similar to a credit card and being formed of a relatively rigid material, said base having a surface with at least one shallow recess therein;
a medication disposed in said recess on said base; and
a removable cover disposed on said base overlying said recess to enclose said medication on said base, said cover being at least partially removable by a user to provide access to said medication in said recess.

14. The personal medication dispensing card according to claim 13, wherein said medication is in powder form and includes a binding agent.

15. The personal medication dispensing card according to claim 13, wherein said medication is in powder form and includes a gelling substance for retaining said medication on said surface.

16. The personal medication dispensing card according to claim 13, wherein said medication substantially fills said recess.

17. The personal medication dispensing card according to claim 13, wherein said base includes a plurality of shallow recesses for containing medication.

18. The personal medication dispensing card according to claim 13, wherein said cover lies substantially flat on said surface of said base.

19. The personal medication dispensing card according to claim 13, wherein said cover is of a pliable material disposed on said base in overlying relation with said recess, said cover being adapted to be peeled back from said recess.

20. The personal medication dispensing card according to claim 13, wherein said cover is adhesively sealed to said base about the periphery of said recess.

21. A method of delivering a medication, said method comprising the steps of:
packaging the medication in a medication dispensing card including
applying the medication to a surface of a base of the card; and
applying a removable cover to the base to enclose the medication on the base; and
dispensing the medication including at least partially removing the cover from the base to expose the medication to a user; and soluably removing the medication from the surface.

22. The method according to claim 21, wherein applying the medication to said surface includes the steps of forming a solution of the medication in powder form and a binding agent, applying the solution to said surface, and drying the solution on said surface.

23. The method according to claim 21, wherein applying the medication to said surface includes the steps of combining the medication in powder form with a gelling agent to form a paste-like substance; and spreading the substance on said surface.

24. The method according to claim 21, wherein said base includes at least one shallow recess, and wherein applying the medication to the base includes substantially filling the recess with the medication.

25. A method of using a card surface to deliver a medication, said method comprising:
providing a card having a surface having applied thereto a medication; and contacting said card surface to remove said medication.

26. The method of claim 25, wherein said contact frictionally removes said medication.

27. The method of claim 25, wherein said contact solubly removes said medication.

28. The method of claim 27, wherein said medication is removed from said card surface by oral contact.

29. The method of claim 25, wherein said card surface includes a shallow recess for said applied medication.

30. The method of claim 25, including providing a cover over said applied medication.

31. A method for achieving a therapeutic effect in a subject, said method comprising:
administrating an effective amount of a compound selected from the group consisting of aspirin, vitamin K, nitroglycerine, an anti-histamine, insulin, sugar, a snake bite antidote, epinephrine, heparin, demetrex, zofran, dramamine, scopolamine, a nicotine replacement medication, pepcid, hydergine, compazine and combinations of said medications, wherein said administration is by contacting a surface of a card to remove medication applied thereto.

32. The method of claim 31, wherein said contact solubly removes said medication from said card surface.

33. The method of claim 32, wherein said medication is removed by oral contact.

34. A dosage administration article comprising:
a card having a surface; and a medication selected from the group consisting of aspirin, vitamin K, nitroglycerine, an anti-histamine, insulin, sugar, a snake bite antidote, epinephrine, heparin, demetrex, zofran, dramamine, scopolamine, a nicotine replacement medication, pepcid, hydergine, compazine and combinations of said medications, said medication present in a therapeutic dosage applied to said surface of said card.

35. The article of claim 34, wherein said medication further includes a pharmaceutically acceptable binder.

36. A therapeutic formulation comprising a medication selected from the group consisting of aspirin, vitamin K, nitroglycerine, an anti-histamine, insulin, sugar, a snake bite antidote, epinephrine, heparin, demetrex, zofran, dramamine, scopolamine, a nicotine replacement medication, pepcid, hydergine, compazine and combinations of said medications, said medication applied to a surface of a card surface.
